(19) United States
(12) Patent Application Publication
(10) Pub. No.: US 2019/0194851 A1
(43) Pub. Date: Jun. 27, 2019

(54) Method of having cleaner laundry

(71) Applicant: Vera Ann Flory, West Alexandria, OH (US)

(72) Inventor: Vera Ann Flory, West Alexandria, OH (US)

(73) Assignee: VA DESIGNS, Ltd., West Alexandria, OH (US)

(21) Appl. No.: 16/350,727

(22) Filed: Dec. 21, 2018

Related U.S. Application Data
(60) Provisional application No. 62/708,859, filed on Dec. 26, 2017.

(51) Int. Cl. D06F 29/00; D06F 33/02

(52) U.S. Cl. CPC D06F 29/00; D06F 2232/06; D06F 33/02

(57) ABSTRACT

This method for cleaner laundry is a preferred method over prior art because it is ecology safer, saves money and energy resources while removing bacteria, bad smells and toxins and generally making life more pleasant. The object of this invention is to have cleaner washed laundry by detoxifying, deodorizing and disinfecting. This method uses an apparatus called Lightning Clean Technology™ with a leak-proof water tank surrounding the washing machine tub to stabilize the tub and to hold the water that is bring changed by Lightening Clean Technology™. This water is then used in the last rinse. This invention makes laundry genuinely clean without using chemicals or too hot of water or steam.

### CROSS REFERENCE TO RELATED APPLICATION

This patent application is related to a Provisional Patent Application filed on Dec. 26, 2017 and granted on Jan. 18, 2018 with country code and number of U.S. No. 62/708,859

### BACKGROUND OF THE INVENTION

### a—Field of Invention

This invention generally relates to a method for having cleaner laundry.

### b—Background

People use smelly, toxic detergents and other chemicals to do their laundry with, which is harmful to the user and to the ecology system. And some people use very hot water or even steam to disinfect, which can fade or shrink their laundry.

### BRIEF SUMMARY OF THE INVENTION

The object of this invention is to have cleaner washed laundry by detoxifying, deodorizing and disinfecting, without using harmful smelly chemicals and/or extremely hot water that can shrink or fade the laundry. Another object of this invention is saving money, energy resources, the quality of the environment and the laundry operator's health, while removing toxins and generally making life more pleasant.

### BRIEF DESCRIPTION OF THE VIEWS OF THE DRAWING

FIG. 1 shows a laundry washing machine. While there are many brands, shapes, and sizes, the door and the control panel is not always located at the same place. This drawing shows a washer with the control panel near the back just as an illustration.

FIG. 2 #1 shows a waterproof container surrounding a washer tub.

FIG. 2 #2 shows the circle where the washer tub could be. While there are many brands, shapes, and sizes, the washer tub can be rotated. This drawing shows a washer tub in a perpendicular position and is shown just for an illustration of how it could be.

FIG. 2 #3 shows the apparatus as taking part of the space between the washer tub and the shroud. The apparatus could be anywhere, even outside the washer, if so desired.

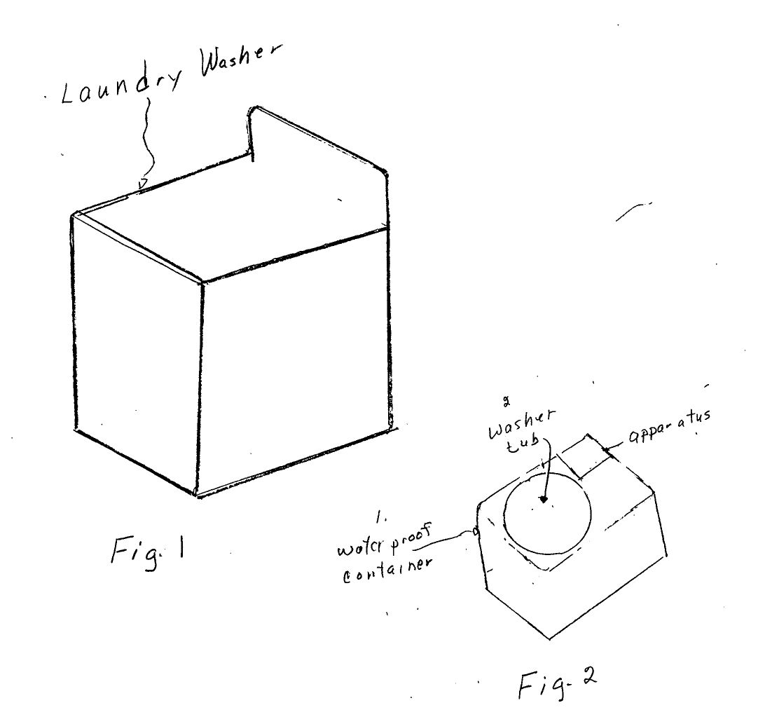

### DETAILED DESCRIPTION OF THE INVENTION

This method for cleaner washed laundry is a preferred method over prior art because it is ecology safer, whereby destroying mold, mildew, tobacco, body odors, fragrances and other offensive smells of which most are toxic. The quality of the environment can be helped by using this invention in removing toxins, bacteria and bad odors and generally making life more pleasant.

This method to have cleaner washed laundry uses an apparatus, called Lightning Clean Technology™, with a washing machine. Said apparatus detoxifies, deodorizes and disinfects, saving money from extra heated laundry (energy resources), as well as saving money replacing ruined clothing.

The method to have cleaner washed laundry uses the apparatus in the washer called Lightning Clean Technology™ to make the water special.

A tank that utilizes extra space between the shroud and tub helps to stabilize the washer and to hold the water that the apparatus makes special.

The apparatus works with the appliance control panel through wiring, radio waves, or other wireless methods and the control panel is programed to use the special water in the last rinse. This method takes away the need for fabric softeners in most areas.

What is claimed is:

1. A method for getting clothes cleaner by using an apparatus called Lightning Clean Technology™ connecting to a washer including a leak-proof tank being made to fit in the vacant space between the tub and shroud of the washer including the programer on the washer being set to run water into the leak-proof tank at the beginning of the wash cycle and running this special water into the tub at the last rinse.
